(19) United States
(12) Patent Application Publication
(10) Pub. No.: US 2012/0041476 A1
(43) Pub. Date: Feb. 16, 2012

(54) CUFF

(76) Inventor: Tung-Yi LIN, Banchiau City (TW)

(21) Appl. No.: 12/854,533

(22) Filed: Aug. 11, 2010

(51) Int. Cl. A61B 17/00

(52) U.S. Cl. 606/202

(57) ABSTRACT

The present invention discloses a cuff which includes a wrap band. A surface of the wrap band is provided respectively with a first Velcro strip and a second Velcro strip made of a different material than the first Velcro strip that the first Velcro strip and the second Velcro strip can be fastened and fixed together. An interior of the wrap band is provided with a bladder which is connected with more than one joint pipe. The joint pipes are connected with a pressure regulation device by which a pressure of the bladder is increased and decreased repeatedly to result in a volume change that wounded tissues and surrounding unwounded tissues are depressurized and pressurized, thereby achieving an effect of reducing a risk of necrosis to the wounded tissues and the surrounding unwounded tissues.

### BACKGROUND OF THE INVENTION

a) Field of the Invention

The present invention relates to a cuff, and more particularly to a cuff which, through a pressure regulation device, repeatedly increases and decreases a pressure of a wrap band bladder to produce a compression effect, and also through the pressure regulation device, configures a pressure increase and a fixed pressure of the bladder, thereby serving as a tourniquet.

b) Description of the Prior Art

A patient suffering from an acute or chronic renal failure, a refractory edema or an internal metabolic abnormality may have a renal disorder that metabolized wastes, toxins or water inside the body cannot be expelled out. Therefore, a course of treatment for hemodialysis needs to be done to expel out the aforementioned wastes, toxins or water.

As vascular sclerosis can be easily formed during the hemodialysis, local compression has to be conducted by a medical personnel to activate blood vessels and surrounding tissues, thereby preventing from speeding up embrittlement of the blood vessels. Nevertheless, a force exerted by the medical personnel varies and hence, it can cause a secondary injury to the patient if the force is exerted exceedingly or insufficiently.

To prevent the patient from bleeding in a large quantity after the hemodialysis, a body part which is pierced by a syringe should be compressed by a proper force through an alcohol cotton ball, at least for 10˜15 min, to stop bleeding.

Allowing the patient who has been accomplished with the hemodialysis to have a convenient tool for hemostasis, most patients now will use the tourniquets for the hemostasis. However, for a conventional latex tourniquet, a latex tube or an elastic band will be used to tie up a body part for the hemostasis, which can only conduct the single-state hemostasis and cannot perform local depressurization or pressurization during the hemostasis. Therefore, wounded tissues and surrounding unwounded tissues can have necrosis as the blood stream has been interrupted too long.

Besides, at the moment, during a surgical operation, a pneumatic tourniquet can be used to reduce the blood stream or completely stop circulation of the blood stream. The aforementioned pneumatic tourniquet includes a pressure regulator, joint pipes and an expandable cuff, wherein the cuff is charged with air through a pneumatic device. When using the pneumatic tourniquet, a limb of the patient is tied up by the cuff and then the cuff is charged with air, thereby achieving the aforementioned effect. Yet, when the cuff of the pneumatic tourniquet is charged with air, the local depressurization or pressurization cannot be done during the surgical operation, either. Therefore, it may still result in the necrosis to muscle tissues as the blood stream has been interrupted too long.

Accordingly, there are still a lot of shortcomings for the prior art described above, which are not good designs and improvement is needed.

### SUMMARY OF THE INVENTION

The primary object of the present invention is to provide a cuff, so as to solve the problem existing in the prior art that it may easily result in the necrosis to the muscle tissues as the blood stream has been interrupted too long.

To achieve the aforementioned object, a technical means of the present invention is to provide a cuff which includes a warp band, a surface of which is provided respectively with a first Velcro strip and a second Velcro strip made of a different material than the first Velcro strip that the first Velcro strip and the second Velcro strip can be fastened and fixed together. An interior of the wrap band is provided with a bladder which is connected with more than one joint pipe, and the join pipes are connected with a pressure regulation device.

Furthermore, the pressure regulation device is a manual pressure regulation device which is composed of an air charging ball and an air discharging device. The air charging ball allows a user to squeeze repeatedly that air can be squeezed into the bladder. The air discharging device is connected on a joint pipe at a front end of the air charging ball and is provided with a depressurization assembly which can operate and adjust a pressure setting and can automatically discharge air when air is charged to exceed the pressure setting.

Furthermore, the manual pressure regulation device and the joint pipes of the wrap band are connected with a pressure gauge.

Furthermore, the pressure regulation device is an automatic pressure regulation device which is composed of an electric air charging motor used to charge air automatically and an electric air discharging device providing the air discharging function.

Furthermore, the automatic pressure regulation device is provided with a timing control device which is used to control an operation time of the automatic pressure regulation device and a pressure control device which is used to configure an air charging pressure.

Furthermore, an end of the wrap band is provided with a clip ring which is transfixed by the other end of the wrap band.

Furthermore, the bladder is designed as plural air sacks which are in a shape of long strips and arranged transversally.

Furthermore, the bladder is designed as plural air sacks which are in a shape of long strips and arranged longitudinally.

Furthermore, the bladder is designed as plural air sacks which are in a shape of long strips and arranged obliquely and separately.

Furthermore, the bladder is designed as two air sacks which are in a shape of long strips and arranged transversally. In a mean time, the long-stripped air sacks are provided with plural air sacks which are arranged longitudinally.

After using the aforementioned structures, through the pressure regulation device of the present invention, the pressure in the bladder of the wrap band can be increased and decreased repeatedly to depressurize and pressurize the wounded tissues and the surrounding unwounded tissues, thereby reducing a risk of the necrosis to the wounded tissues and the surrounding unwounded tissues.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a structure schematic view of a first embodiment of a cuff of the present invention.

FIG. 2 shows a structure schematic view of a first embodiment of a bladder in the cuff of the present invention.

FIG. 3 shows a structure schematic view of a second embodiment of the bladder in the cuff of the present invention.

FIG. 4 shows a structure schematic view of a third embodiment of the bladder in the cuff of the present invention.

FIG. 5 shows a structure schematic view of a fourth embodiment of the bladder in the cuff of the present invention.

FIG. 6 shows a structure schematic view of a second embodiment of the cuff of the present invention.

FIG. 7 shows a structure schematic view of a third embodiment of the cuff of the present invention.

### DETAILED DESCRIPTION OF THE PREFERRED EMBODIMENTS

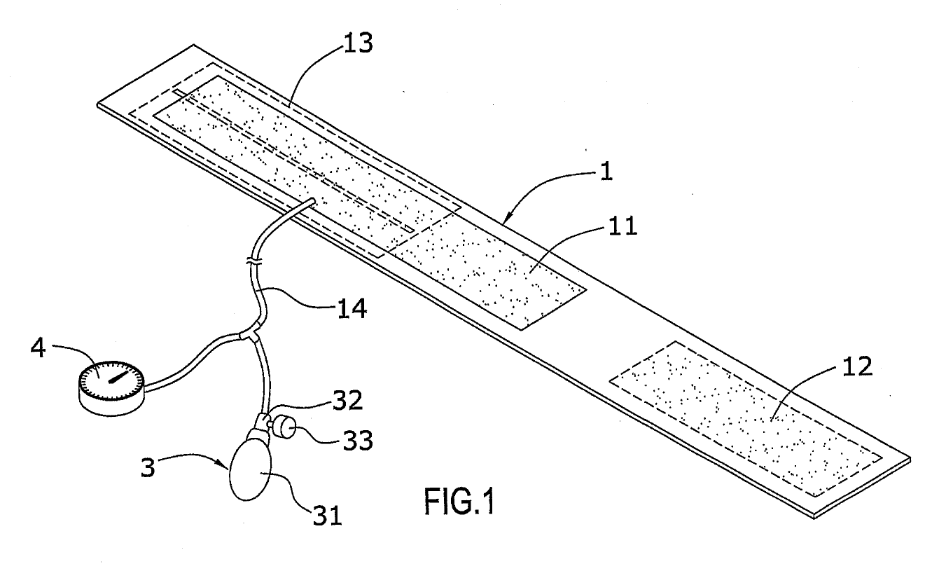

Referring to FIG. 1, it shows a structure schematic view of a first embodiment of a cuff of the present invention, comprising a wrap band 1, two side surfaces of which are provided respectively with a first Velcro strip 11 and a second Velcro strip 12 made of a different material than the first Velcro strip 11 that the first Velcro strip 11 and the second Velcro strip 12 can be fastened and fixed together. An interior of the wrap band 1 is provided with a bladder 13 which is an arrangement of plural air sacks in a shape of long strips. The bladder 13 is extended with at least one joint pipe 14 which can be interconnected with a pressure regulation device. In the present embodiment, the pressure regulation device is a manual pressure regulation device 3 which is constituted by an air charging ball 31 and an air discharging device 32. The air charging ball 31 provides a user to squeeze repeatedly that air can be squeezed into the bladder 13, thereby increasing the pressure inside the bladder 13. The air discharging device 32, whereas, is connected to a joint pipe 14 at a front end of the air charging ball 31 and is provided with a depressurization assembly 33. The user can operate the depressurization assembly 33 to expel out air in the bladder 13, thereby reducing the pressure inside the bladder 13 or adjusting a pressure setting. When air is charged to exceed the pressure setting, air will be discharged automatically to avoid over-pressurization. On the other hand, the manual pressure regulation device 3 and the joint pipe 14 of the wrap band 1 can be connected with a pressure gauge 4 which is able to display the pressure inside the bladder 13, allowing the user to control the pressure inside the bladder 13 by himself or herself, through the pressure displayed by the pressure gauge 4, thereby repeatedly increasing and decreasing the pressure of the bladder 13 to depressurize and pressurize wounded tissues and surrounding unwounded tissues, through a volume change of the bladder 13 due to the pressure change, so as to reduce a risk of necrosis to the wounded tissues and the surrounding unwounded tissues.

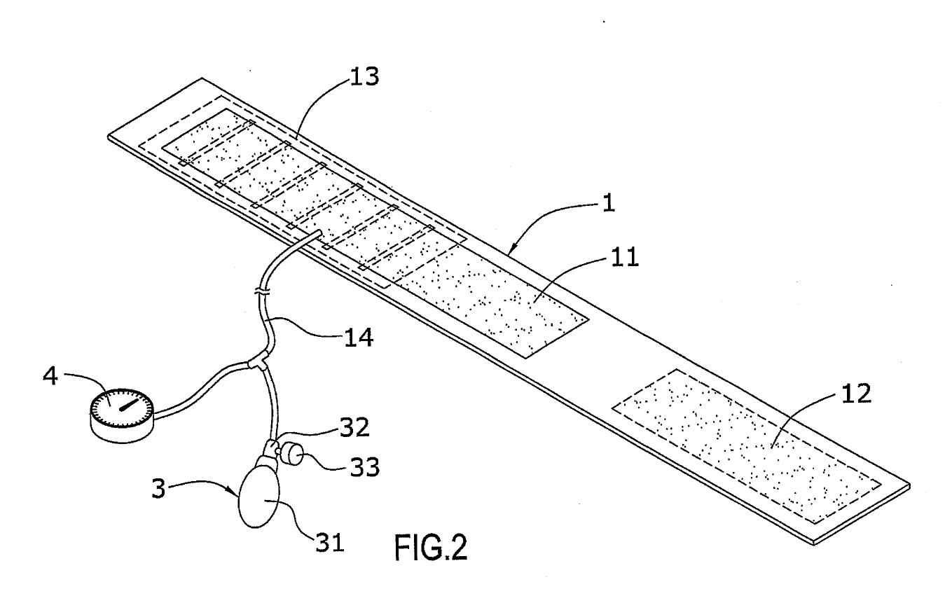

Referring to FIG. 2, it shows a structure schematic view of a first embodiment of the bladder in the cuff of the present invention. In the present embodiment, the structures and layout of the wrap band 1, the joint pipes 14 and the manual pressure regulation device 3 are all the same as those in FIG. 1, thus no further description is disclosed. The difference lies in that the bladder 13 is a longitudinal arrangement of plural air sacks in a shape of long strips, allowing the bladder 13 to be arranged in the shape of long strips after being charged with air. When the bladder 13 is subjected to the repeated increase and decrease of pressure by the manual pressure regulation device 3, a longitudinal compression effect can be achieved to the bladder 13.

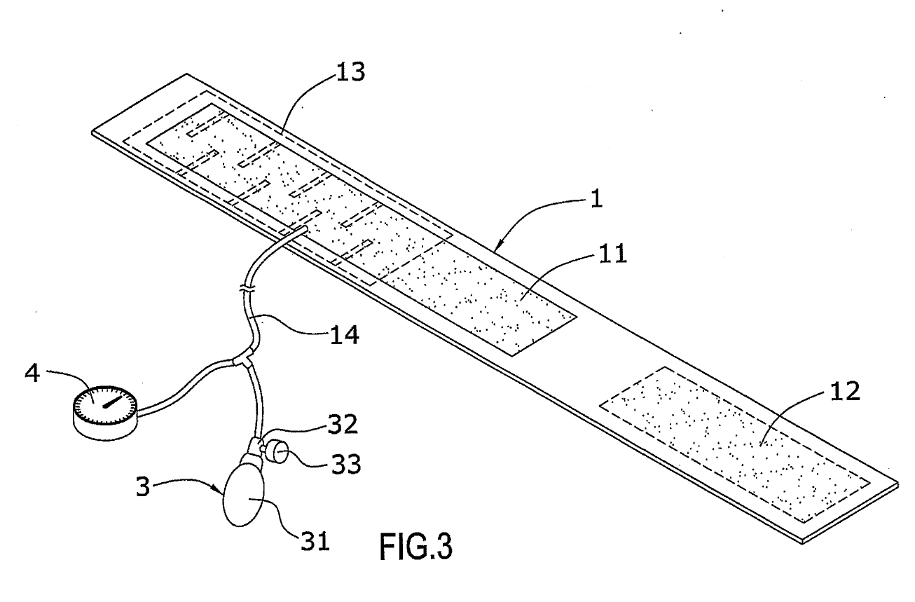

Referring to FIG. 3, it shows a structure schematic view of a second embodiment of the bladder in the cuff of the present invention. In the present embodiment, the structures and layout of the wrap band 1, the joint pipes 14 and the manual pressure regulation device 3 are all the same as those in FIG. 1, thus no further description is disclosed. The difference lies in that the bladder 13 is a longitudinal arrangement of plural air sacks which are separated in intervals alternately. When the bladder 13 is subjected to the repeated increase and decrease of pressure by the manual pressure regulation device 3, the bladder 13 can be expanded or diminished longitudinally by intervals, thereby being able to compress different body parts of the user.

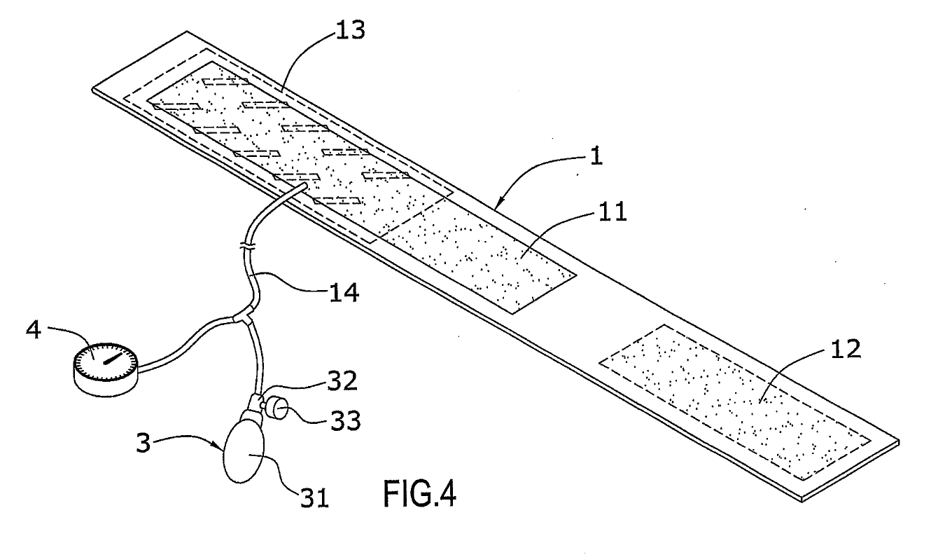

Referring to FIG. 4, it shows a structure schematic view of a third embodiment of the bladder of the cuff of the present invention. In the present embodiment, the structures and layout of the wrap band 1, the joint pipes 14 and the manual pressure regulation device 3 are all the same as those in FIG. 1, thus no further description is disclosed. The difference lies in that the bladder 13 is an oblique arrangement of plural air sacks which are separated in intervals alternately, allowing the bladder 13 to be expanded or diminished obliquely by intervals when the bladder 13 is subjected to the repeated increase and decrease of pressure by the manual pressure regulation device 3. Thus, another different body part of the user can be compressed.

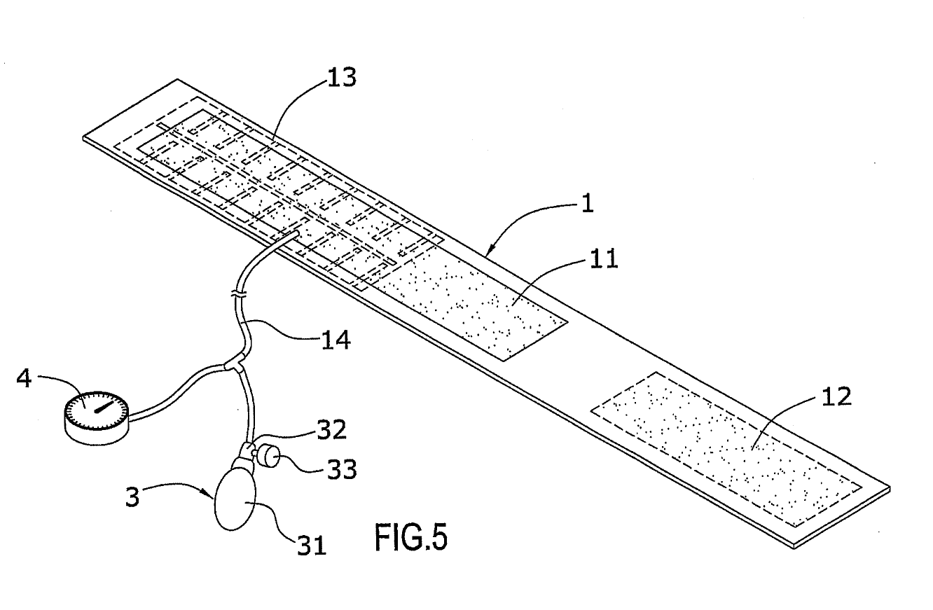

Referring to FIG. 5, it shows a structure schematic view of a fourth embodiment of the bladder in the cuff of the present invention. In the present embodiment, the structures and layout of the wrap band 1, the joint pipes 14 and the manual pressure regulation device 3 are all the same as those in FIG. 1, thus no further description is disclosed. The difference lies in that the bladder 13 is a transversal arrangement of two air sacks which are in a shape of long strips and are provided with plural longitudinally arranged air sacks, allowing the bladder 13 to be arranged regularly in the transversal and longitudinal directions when the bladder 13 is subjected to the repeated increase and decrease of pressure by the manual pressure regulation device 3. Therefore, another different body part of the user can be compressed.

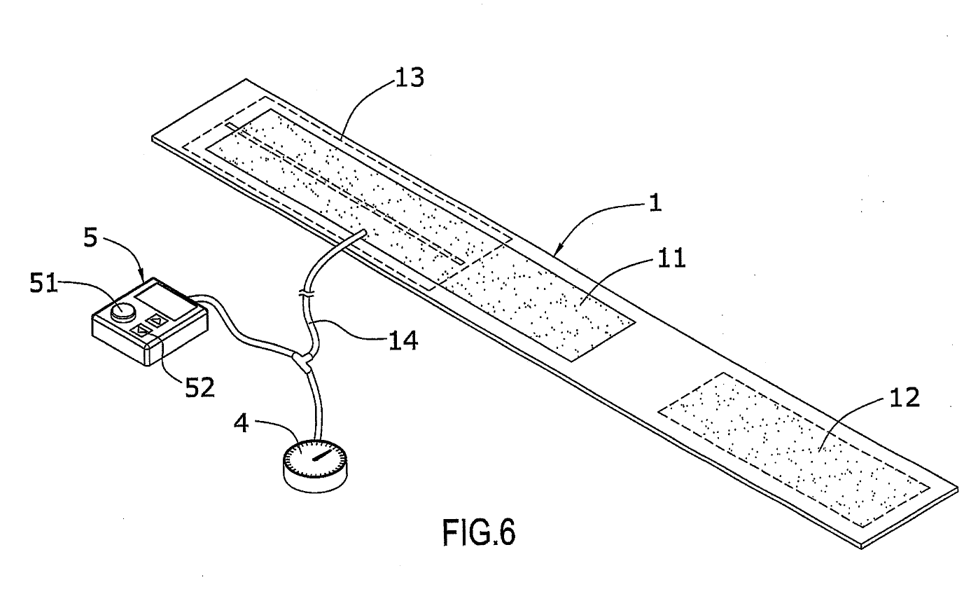

Referring to FIG. 6, it shows a structure schematic view of a second embodiment of the cuff of the present invention, comprising a wrap band 1, two side surfaces of which are provided respectively with a first Velcro strip 11 and a second Velcro strip 12 made of a different material than the first Velcro strip 11 that the first Velcro strip 11 and the second Velcro strip 12 can be fastened and fixed together. An interior of the wrap band 1 is provided with a bladder 13 which is an arrangement of plural air sacks in a shape of long strips or a longitudinal arrangement of plural air sacks separated in intervals alternately, an oblique arrangement of plural air sacks separated in intervals alternately or a transversal arrangement of two air sacks in a shape of long strips and having plural longitudinally arranged air sacks, as described in the aforementioned embodiments. The bladder 13 is extended with more than one joint pipe 14 which is interconnected with a pressure regulation device. In the present embodiment, the pressure regulation device is an automatic pressure regulation device 5 which is constituted by an electric air charging motor and an electric air discharging device (not shown in the drawing) inside the pressure regulation device 5. An automatic air charging effect is achieved through the electric air charging motor, whereas the electric air discharging device provides the air discharging function. In addition, the automatic pressure regulation device 5 is provided with a timing control device 51 and a pressure control device 52. The timing control device 51 can control an operation time of the automatic pressure regulation device 5 and the pressure control device 52 can be used to configure an air charging pressure. Through mutual collaboration of the aforementioned components, the pressure of the bladder 13 can be increased and decreased repeatedly to depressurize and pressurize the wounded tissues and the surrounding unwounded tissues through the volume change of the bladder 13 due to the pressure change, thereby reducing the risk of the necrosis to the wounded tissues and the surrounding unwounded tissues. Besides, when the automatic pressure regulation device 5 only activates the pressure control device 52, the electric air charging motor will increase the pressure inside the bladder 13 to the pressure that is set up by the pressure control device 52, thereby achieving the function of tourniquet and improving a scope and a field of application.

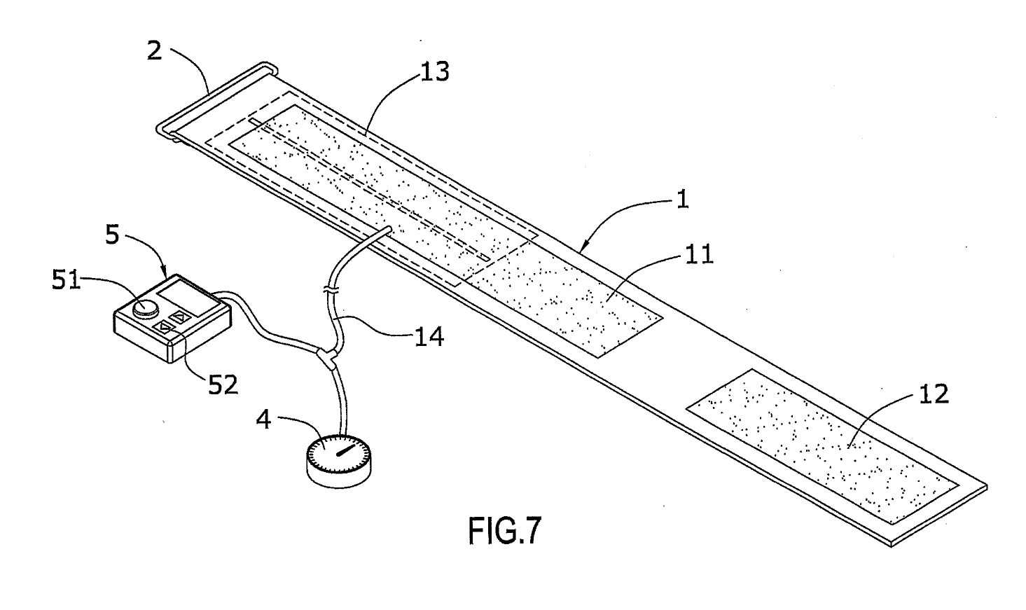

Referring to FIG. 7, it shows a structure schematic view of a third embodiment of the cuff of the present invention. In the present embodiment, the structures are the same as those of the second embodiment in FIG. 6, thus no further description is disclosed. The difference lies in that an end of the wrap band 1 can be provided with a clip ring 2 which is transfixed by the other end of the wrap band 1. A same side surface of the wrap band 1 is provided respectively with a first Velcro strip 11 and a second Velcro strip 12 made of a different material than the first Velcro strip 11 that the first Velcro strip 11 and the second Velcro strip 12 can be fastened and fixed together, thereby allowing the user to adjust wrapping depending on a thickness of arm circumference when operating and using the wrap band 1.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. A cuff comprising a wrap band, a surface of which is provided respectively with a first Velcro strip and a second Velcro strip made of a different material than the first Velcro strip that the first Velcro strip and the second Velcro strip are fastened and fixed together, an interior of the wrap band being provided with a bladder which is connected with more than one joint pipe which is connected with a pressure regulation device.

2. The cuff according to claim 1, wherein the pressure regulation device is a manual pressure regulation device which is constituted by an air charging ball and an air discharging device, with the air charging ball providing a user to squeeze repeatedly that air is squeezed into the bladder, the air discharging device being connected with a joint pipe at a front end of the air charging ball and being provided with a depressurization assembly which operates and adjusts a pressure setting and discharges air automatically when air is charged to exceed the pressure setting.

3. The cuff according to claim 2, wherein the manual pressure regulation device and the joint pipes of the wrap band are connected with a pressure gauge.

4. The cuff according to claim 1, wherein the pressure regulation device is an automatic pressure regulation device which is constituted by an electric air charging motor used to charge air automatically and an electric air discharging device providing the air discharging function.

5. The cuff according to claim 4, wherein the automatic pressure regulation device is provided with a timing control device which is used to control an operation time of the automatic pressure regulation device and a pressure control device which is used to configure an air charging pressure.

6. The cuff according to claim 1, wherein an end of the wrap band is provided with a clip ring which is transfixed by the other end of the wrap band.

7. The cuff according to claim 1, wherein the bladder is a transversal arrangement of plural air sacks which are in a shape of long strips.

8. The cuff according to claim 1, wherein the bladder is a longitudinal arrangement of plural air sacks which are in a shape of long strips.

9. The cuff according to claim 1, wherein the bladder is an oblique arrangement of plural air sacks which are in a shape of long strips and separated in intervals alternately.

10. The cuff according to claim 1, wherein the bladder is a transversal arrangement of two air sacks which are in a shape of long strips and are provided with plural longitudinally arranged air sacks.
